(19) United States
(12) Patent Application Publication
(10) Pub. No.: US 2023/0028751 A1
(43) Pub. Date: Jan. 26, 2023

(54) ARM SUPPORT

(71) Applicant: Brad Hagen, Huntsville, TX (US)

(72) Inventor: Brad Hagen, Huntsville, TX (US)

(21) Appl. No.: 17/693,339

(22) Filed: Mar. 12, 2022

Related U.S. Application Data
(60) Provisional application No. 63/137,120, filed on Jan. 13, 2021.

(51) Int. Cl. A61F 5/37; A63B 71/08

(52) U.S. Cl. CPC A61F 5/373; A63B 71/08; A63B 2243/0054

(57) ABSTRACT

Various embodiments of an arm support are disclosed. An arm support may include a support member having a forearm section and an elbow section. An arm support may have a plurality of opposed longitudinal slots adapted for engagement with releasably attachable straps for securing the arm support to a person's forearm. A support member may bend upwardly at a transition point between the forearm section and the elbow section. A forearm pad may be attached to the forearm section. An elbow pad may be attached to the elbow section. The elbow pad may include an upwardly inclined surface. A support member may include a concave upper surface. An arm support may be useful to prevent hyperflexion or hyperextension, such as hyperextension of the elbow joint of a bowler.

### RELATED APPLICATIONS

This application claims the benefit of U.S. Provisional Application No. 63/137,120, filed Jan. 13, 2021, the contents of which are fully incorporated herein by reference.

### BACKGROUND OF THE INVENTIONS

### 1. Field of the Inventions

The present inventions generally pertain to athletic equipment, and more particularly to arm supports to prevent hyperflexion or hyperextension, such as hyperextension of the elbow joint of a bowler.

### 2. Description Of The Related Art

Hyperflexion is the flexion movement of a muscle that extends beyond the body's normal range of motion. In hyperflexion, a joint is bent beyond its normal limits and can cause injury of varying degrees and severity. When the joint is hyper flexed, it is moved beyond the maximum safe flexion. Such extreme movement can potentially result in injury issues to the opposing ligaments, tendons, and muscles.

There is a need, especially in the sport of bowling, for an arm support the purpose of which is to avoid the hyperflexion injury thus preventing the short term and long term effects of training as an elite athlete in bowling. There is a need for such an arm support to prevent injury and prevent further damage such as: swelling, bruising, ligament discomfort, stiffness, bowler's elbow, overuse, range of motion through the shoulder, muscle spasms, physical therapy and/or surgery.

### SUMMARY OF THE INVENTIONS

In one aspect, the present inventions may include an arm support comprising a support member having a forearm section and an elbow section; at least one releasably attachable strap attached to the support member; a forearm pad attached to a first surface of the forearm section; and an elbow pad attached to a first surface of the elbow section. Another feature of this aspect of the present inventions may be that the arm support may further include a first longitudinal slot disposed adjacent a first edge of the support member; a second longitudinal slot disposed adjacent a second edge of the support member opposite from the first longitudinal slot; a third longitudinal slot disposed adjacent the first edge of the support member and between a first end of the support member and the first longitudinal slot; and a fourth longitudinal slot disposed adjacent the second edge of the support member opposite from the third longitudinal slot. Another feature of this aspect of the present inventions may be that the elbow pad includes an upwardly inclined surface extending from a height adjacent a height of the forearm pad to a rear height of the elbow pad. Another feature of this aspect of the present inventions may be that the rear height of the elbow pad is in the range from 13 mm to 38 mm. Another feature of this aspect of the present inventions may be that the support member includes a transitional concave bend from the forearm section to the elbow section, wherein the elbow section extends upwardly away from the forearm section. Another feature of this aspect of the present inventions may be that the support member includes a concave upper surface. Another feature of this aspect of the present inventions may be that the first surface of the forearm section is a concave surface.

In another aspect, the present inventions may include an arm support comprising: a support member having a forearm section and an elbow section; at least one releasably attachable strap attached to the support member; a forearm pad attached to a first surface of the forearm section; and an elbow pad attached to a first surface of the elbow section, the elbow pad including an upwardly inclined surface extending from a height adjacent a height of the forearm pad to a rear height of the elbow pad. Another feature of this aspect of the present inventions may be that the rear height of the elbow pad is in the range from 13 mm to 38 mm. Another feature of this aspect of the present inventions may be that the arm support may further include a first longitudinal slot disposed adjacent a first edge of the support member; a second longitudinal slot disposed adjacent a second edge of the support member opposite from the first longitudinal slot; a third longitudinal slot disposed adjacent the first edge of the support member and between a first end of the support member and the first longitudinal slot; and a fourth longitudinal slot disposed adjacent the second edge of the support member opposite from the third longitudinal slot. Another feature of this aspect of the present inventions may be that the support member includes a transitional concave bend from the forearm section to the elbow section, wherein the elbow section extends upwardly away from the forearm section. Another feature of this aspect of the present inventions may be that the support member includes a concave upper surface. Another feature of this aspect of the present inventions may be that the first surface of the forearm section is a concave surface.

In still another aspect, the present inventions may include an arm support comprising: a support member having a forearm section and an elbow section; the support member including a transitional bend from the forearm section to the elbow section, the elbow section extending upwardly away from the forearm section; a first longitudinal slot disposed adjacent a first edge of the support member; a second longitudinal slot disposed adjacent a second edge of the support member opposite from the first longitudinal slot; a third longitudinal slot disposed adjacent the first edge of the support member and between a first end of the support member and the first longitudinal slot; and a fourth longitudinal slot disposed adjacent the second edge of the support member opposite from the third longitudinal slot. Another feature of this aspect of the present inventions may be that the arm support may further include a first releasably attachable strap adapted for engagement with the first longitudinal slot and the second longitudinal slot, and a second releasably attachable strap adapted for engagement with the third longitudinal slot and the fourth longitudinal slot. Another feature of this aspect of the present inventions may be that the arm support may further include a forearm pad attached to a first surface of the forearm section; and an elbow pad attached to a first surface of the elbow section. Another feature of this aspect of the present inventions may be that the elbow pad includes an upwardly inclined surface extending from a height adjacent a height of the forearm pad to a rear height of the elbow pad. Another feature of this aspect of the present inventions may be that the rear height of the elbow pad is in the range from 13 mm to 38 mm. Another feature of this aspect of the present inventions may be that the support member includes a concave upper surface. Another feature of this aspect of the present inventions may be that the first surface of the forearm section is a concave surface.

Other features, aspects and advantages of the present inventions will become apparent from the following discussion and detailed description.

### BRIEF DESCRIPTION OF THE DRAWINGS

FIG. 1 is a perspective view of a specific embodiment of an arm support according to the present inventions, and shown secured to a person's forearm.

FIG. 2 is another perspective view of the arm support and forearm as shown in FIG. 1, but from a different angle.

FIG. 3 is a perspective view of a specific embodiment of an arm support of the present inventions.

FIG. 4 is an end view of the arm support shown in FIG. 3.

FIG. 5 is a perspective view of a rear side of the arm support shown in FIGS. 3 and 4.

FIG. 6 is a top view of the arm support shown in FIGS. 3-5.

FIG. 7 is a side view of the arm support shown in FIGS. 3-6.

FIG. 8 is a rear view of the arm support shown in FIGS. 3-7.

FIG. 9 is another perspective view of the arm support shown in FIGS. 3-8.

FIG. 10 is another perspective view of the arm support shown in FIGS. 3-9.

FIG. 11 is a side view of a specific embodiment of an arm support according to the present inventions, and illustrating an elbow pad having a first height or thickness.

FIG. 12 is a side view of an arm support similar to FIG. 11, but showing an elbow pad having a second or intermediate height or thickness.

FIG. 13 is a side view of an arm support similar to FIGS. 11 and 12, but showing an elbow pad having a third or relatively large height or thickness.

FIG. 14 is a top view of a specific embodiment of an arm support with fastener straps extended to one side of the arm support.

FIG. 15 is a top view of foam pad inserts or pads that may be used in connection with an arm support of the present inventions.

FIG. 16 is a top view of an arm support in accordance with the present inventions, without fastening straps attached thereto.

FIG. 17 is a top view of an arm support in accordance with the present inventions, and showing fastener straps extended to one side of the arm support.

While the inventions will be described in connection with the preferred embodiments, it will be understood that the scope of protection is not intended to limit the inventions to those embodiments. On the contrary, the scope of protection is intended to cover all alternatives, modifications, and equivalents as may be included within the spirit and scope of the inventions as defined by the appended claims.

### DETAILED DESCRIPTION OF THE INVENTION

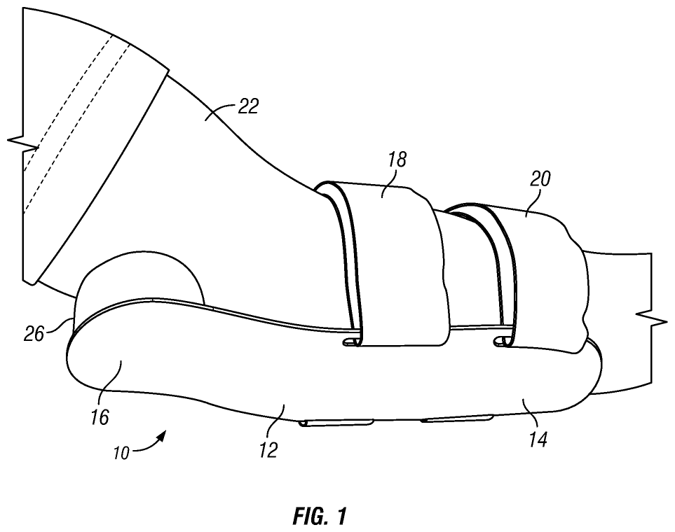

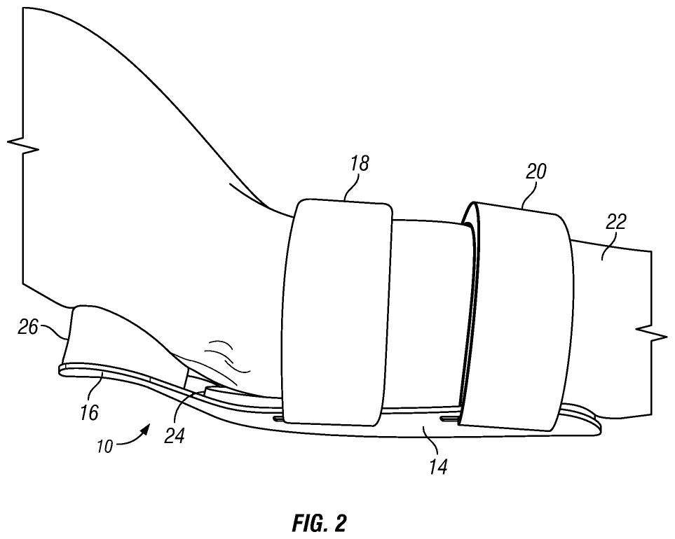

Referring to the drawings in detail, wherein like numerals denote identical elements throughout the several views, and referring initially to FIGS. 1 and 2, in a specific embodiment, the present inventions may encompass an arm support 10 comprising a support member 12 having a forearm section 14 and an elbow section 16. The forearm section 14 may include a plurality of straps 18 and 20 adapted to secure the arm support 10 to a person's arm 22, such as with the use of Velcro® fasteners. The support member 12 may include longitudinal slots 15, 17, 19, and 21 for engagement with the straps 18 and 20. Slots 15 and 19 may be disposed on opposed edges of the support member 12 and adapted for engagement with the strap 18. Slots 17 and 21 may be disposed on opposed edges of the support member and adapted for engagement with the strap 20. The slots 17 and 21 may be disposed adjacent a first or distal end of the forearm section 14 of the support member 12. The slots 15 and 19 may be disposed approximately midway between opposed ends of the support member 12. The slots 15 and 19 may also be disposed near a transition point or bend in the support member 12 where the forearm section 14 transitions to the elbow section 16.

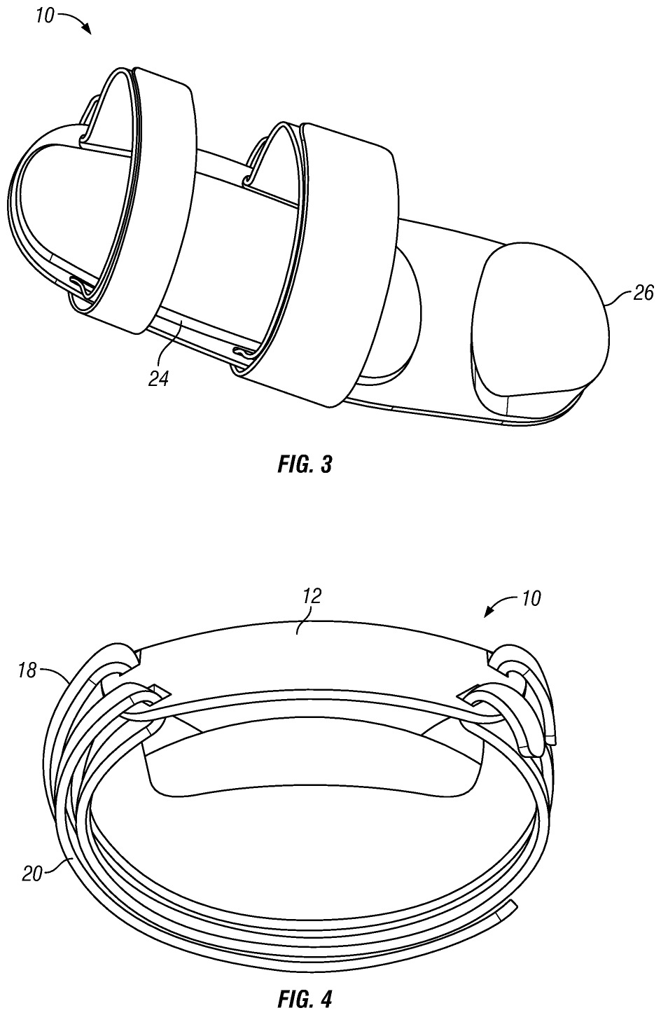

As can be seen in FIG. 2, the forearm section 14 may include a forearm pad 24 attached to the forearm section 14, and an elbow pad 26 attached to the elbow section 16. In a specific embodiment, the support member 12 may be provided with a generally concave cross-sectional profile to generally conform to the shape of an arm 22 (see, e.g., FIG. 4). As best seen in FIG. 2, in a specific embodiment, the elbow pad 26 may be much thicker than the forearm pad 24. In a specific embodiment, the elbow pad 26 may have an inclined surface angled upwardly from the front to the back of the elbow pad, such as to form a contoured ramp adapted for mating engagement with the lower tricep.

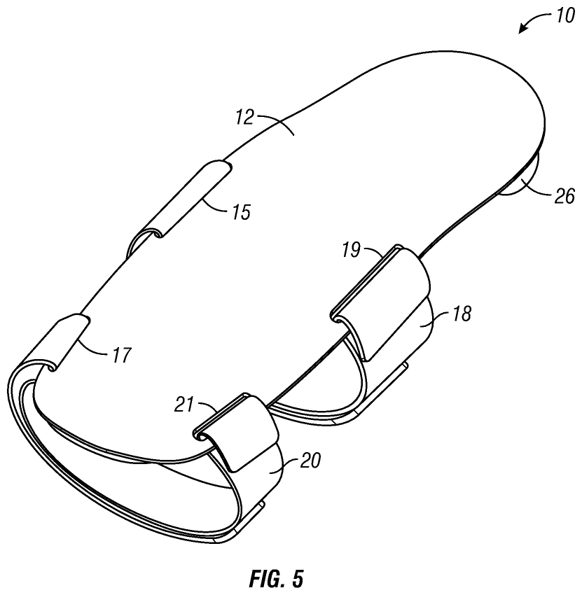

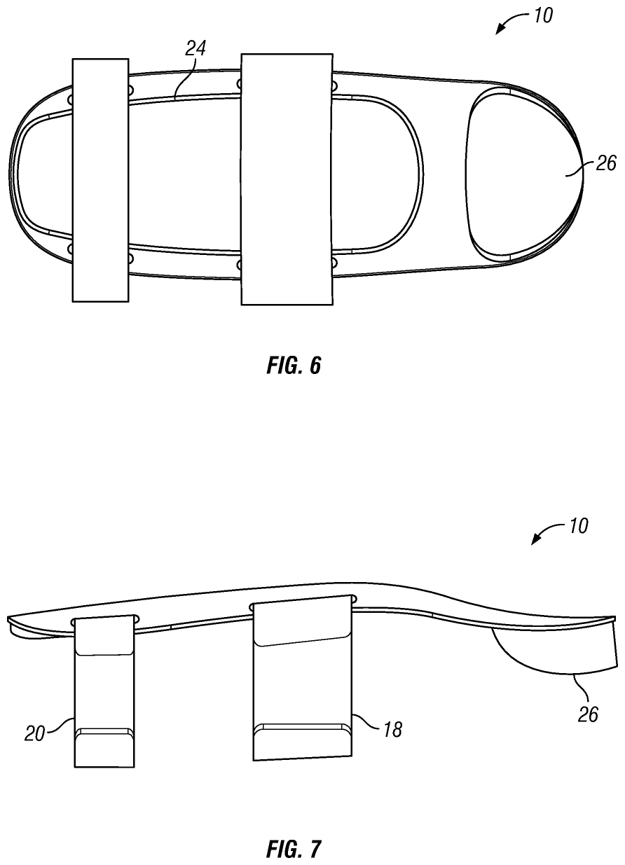

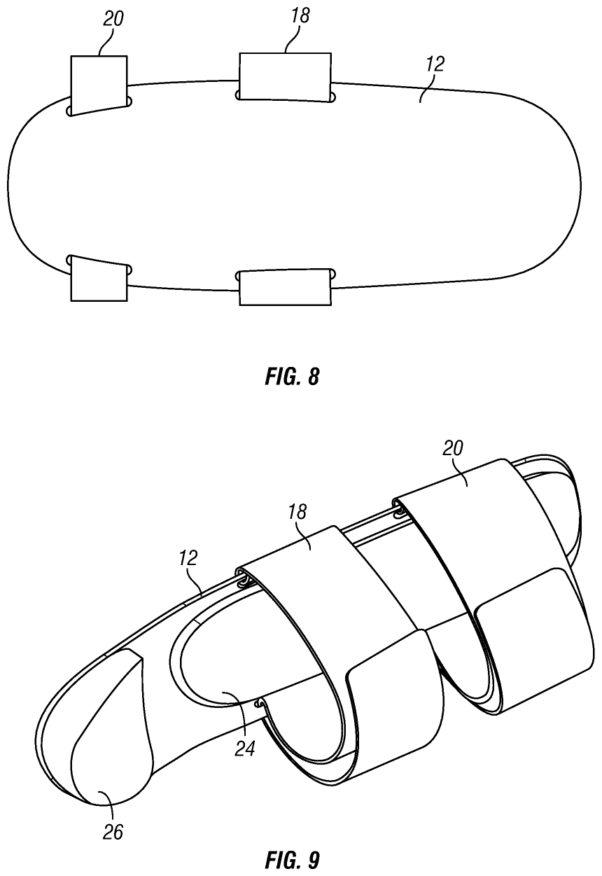

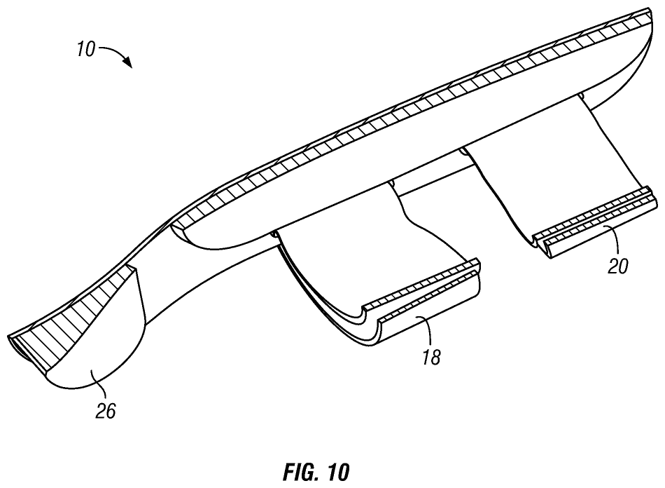

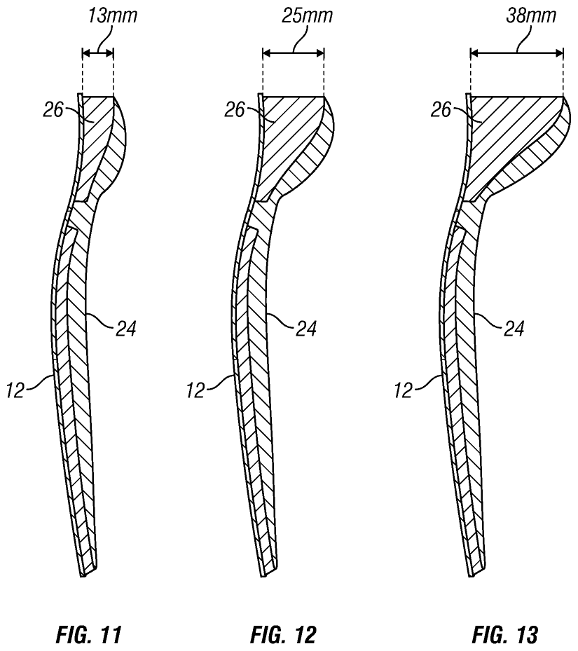

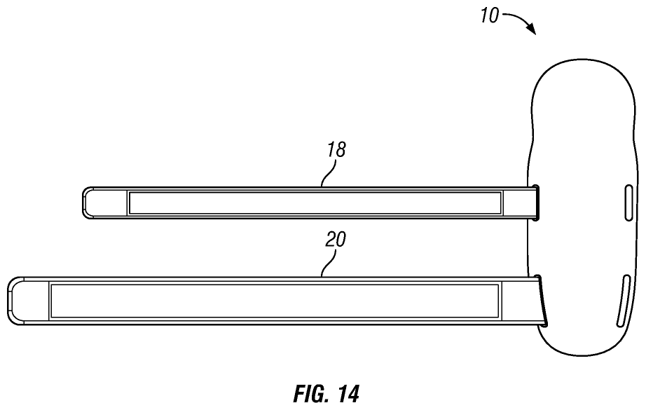

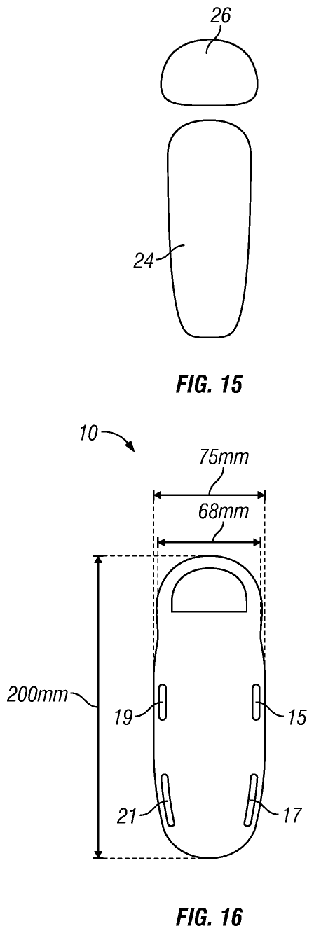

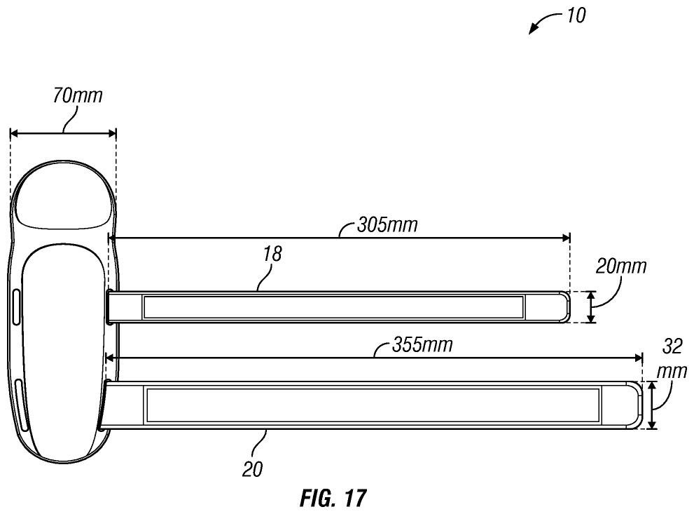

Specific embodiments of the present inventions are also illustrated in FIGS. 3-17. With reference to FIGS. 11, 12 and 13, it can be seen that various example embodiments of elbow pads 26 or pad inserts 28, 30, and 32 are contemplated, and illustrate that the side profile of the elbow pad 26 can be provided with various contours, angles of inclination and thicknesses at their highest or thickest point. Such thicknesses can range from relatively small, such as 13 mm for example as shown in FIG. 11, to intermediate, such as 25 mm for example as shown in FIG. 12, to large, such as 38 mm for example as shown in FIG. 13. The elbow pads 26 may be permanently affixed to the support member 12, or provided as pad inserts 28, 30 and 32 of various sizes adapted for releasable engagement with the support member 12.

It has been discovered that, when the arm support 10 is in use, such as shown in FIGS. 1 and 2, the arm support 10 functions to prevent hyperextension of the elbow. The arm support 10 further functions to prevent injury and prevent further damage such as: swelling, bruising, ligament discomfort, soreness, stiffness, Bowler's elbow, overuse, range of motion through the shoulder, muscle spasms, physical therapy and/or surgery. While the arm support 10 has a preventive function, in addition, by keeping the arm in a straighter linear position the arm support 10 also serves as a leverage tool therefore increasing a bowler's potential ball hook. Another benefit of the arm support 10 is that it is compliant with collegiate and professional bowling rules and regulations.

Another feature of the present inventions is that the arm support 10 does not affect bowling fitting anywhere as much as a wrist support may do so around the wrist or any contraption that restricts the wrist or fingers. Additionally, use of the arm support 10 will result in a more accurate fitting. In addition, if the athlete does not have any wrist issues, the arm support 10 will outperform any wrist support on the market all due to the arm/elbow area being in a more neutral/leverage position.

The arm support 10 is intended to be universal as well as a great opportunity originally for those who suffer from major carrying angles and hyper extension, which tends to occur mostly in females, as well as senior citizens. The arm support 10 can be a tool for all demographics no matter their age to provide preventive, training, and/or performance functions. Very few supports on the market cover all three of these factors without influencing fitting. The arm support 10 is useful to professional and amateur bowlers seeking to correct hyperextension in arms and potentially increase performance during practice and competition.

It is to be understood that the inventions disclosed herein are not limited to the exact details of construction, operation, exact materials or embodiments shown and described. Although specific embodiments of the inventions have been described, various modifications, alterations, alternative constructions, and equivalents are also encompassed within the scope of the inventions. Although the present inventions may have been described using a particular series of steps, it should be apparent to those skilled in the art that the scope of the present inventions is not limited to the described series of steps. The specification and drawings are, accordingly, to be regarded in an illustrative rather than a restrictive sense. It will be evident that additions, subtractions, deletions, and other modifications and changes may be made thereunto without departing from the broader spirit and scope of the inventions as set forth herein.

1. An arm support comprising:
a support member having a forearm section and an elbow section;
at least one releasably attachable strap attached to the support member;
a forearm pad attached to a first surface of the forearm section; and
an elbow pad attached to a first surface of the elbow section.

2. The arm support of claim 1, further including:
a first longitudinal slot disposed adjacent a first edge of the support member;
a second longitudinal slot disposed adjacent a second edge of the support member opposite from the first longitudinal slot;
a third longitudinal slot disposed adjacent the first edge of the support member and between a first end of the support member and the first longitudinal slot; and
a fourth longitudinal slot disposed adjacent the second edge of the support member opposite from the third longitudinal slot.

3. The arm support of claim 1, wherein the elbow pad includes an upwardly inclined surface extending from a height adjacent a height of the forearm pad to a rear height of the elbow pad.

4. The arm support of claim 3, wherein the rear height of the elbow pad is in the range from 13 mm to 38 mm.

5. The arm support of claim 1, wherein the support member includes a transitional concave bend from the forearm section to the elbow section, wherein the elbow section extends upwardly away from the forearm section.

6. The arm support of claim 1, wherein the support member includes a concave upper surface.

7. The arm support of claim 1, wherein the first surface of the forearm section is a concave surface.

8. An arm support comprising:
a support member having a forearm section and an elbow section;
at least one releasably attachable strap attached to the support member;
a forearm pad attached to a first surface of the forearm section; and
an elbow pad attached to a first surface of the elbow section, the elbow pad including an upwardly inclined surface extending from a height adjacent a height of the forearm pad to a rear height of the elbow pad.

9. The arm support of claim 8, wherein the rear height of the elbow pad is in the range from 13 mm to 38 mm.

10. The arm support of claim 8, further including:
a first longitudinal slot disposed adjacent a first edge of the support member;
a second longitudinal slot disposed adjacent a second edge of the support member opposite from the first longitudinal slot;
a third longitudinal slot disposed adjacent the first edge of the support member and between a first end of the support member and the first longitudinal slot; and
a fourth longitudinal slot disposed adjacent the second edge of the support member opposite from the third longitudinal slot.

11. The arm support of claim 8, wherein the support member includes a transitional concave bend from the forearm section to the elbow section, wherein the elbow section extends upwardly away from the forearm section.

12. The arm support of claim 8, wherein the support member includes a concave upper surface.

13. The arm support of claim 8, wherein the first surface of the forearm section is a concave surface.

14. An arm support comprising:
a support member having a forearm section and an elbow section;
the support member including a transitional bend from the forearm section to the elbow section, the elbow section extending upwardly away from the forearm section;
a first longitudinal slot disposed adjacent a first edge of the support member;
a second longitudinal slot disposed adjacent a second edge of the support member opposite from the first longitudinal slot;
a third longitudinal slot disposed adjacent the first edge of the support member and between a first end of the support member and the first longitudinal slot; and
a fourth longitudinal slot disposed adjacent the second edge of the support member opposite from the third longitudinal slot.

15. The arm support of claim 14, further including a first releasably attachable strap adapted for engagement with the first longitudinal slot and the second longitudinal slot, and a second releasably attachable strap adapted for engagement with the third longitudinal slot and the fourth longitudinal slot.

16. The arm support of claim 14, further including:
a forearm pad attached to a first surface of the forearm section; and
an elbow pad attached to a first surface of the elbow section.

17. The arm support of claim 14, wherein the elbow pad includes an upwardly inclined surface extending from a height adjacent a height of the forearm pad to a rear height of the elbow pad.

18. The arm support of claim 17, wherein the rear height of the elbow pad is in the range from 13 mm to 38 mm.

19. The arm support of claim 14, wherein the support member includes a concave upper surface.

20. The arm support of claim 14, wherein the first surface of the forearm section is a concave surface.
